(19) United States
(12) Patent Application Publication
(10) Pub. No.: US 2005/0181513 A1
(43) Pub. Date: Aug. 18, 2005

(54) Methods and compositions for assessing a sample by MAILDI mass spectrometry

(76) Inventors: Viorica Lopez-Avila, Cupertino, CA (US); David L. Hirschberg, Menlo Park, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/782,269

(22) Filed: Feb. 18, 2004

(51) Int. Cl. G06F 19/00; G01N 33/48; G01N 33/50; G01N 33/00

(52) U.S. Cl. 436/86; 250/288; 702/19

(57) ABSTRACT

The invention provides methods for preparing a MALDI sample plate. In general, the methods involve contacting a sample with an array of features containing capture agents that specifically bind to analytes, processing any analytes bound to the capture agents for MALDI analysis, and transferring the processed analytes to a MALDI sample plate. Also provided is a system for preparing a MALDI sample plate, containing an automatic fluid delivery device that is fluidically connected to a sample, and also to MALDI processing agents. In certain embodiments, the analytes present on the prepared subject MALDI sample plate may be evaluated by mass spectrometry. Kits and other compositions are provided for performing the subject methods. The subject invention finds use in methods of simultaneously assessing the presence of several analytes in a single sample, and, as such, the invention finds use in a variety of different medical, research and proteomics applications.

### FIELD OF THE INVENTION

The field of this invention is sample analysis, particularly the analysis of complex samples using MALDI mass spectrometry.

### BACKGROUND OF THE INVENTION

Straightforward and reliable methods for simultaneously analyzing several constituents of a complex sample are extremely desirable. For example, it is desirable to determine the relative amounts of several pre-determined analytes, e.g., proteins, in blood and other bodily fluids, in medical diagnostics and other fields. However, current methodologies for sample analysis are impractical for such uses.

For example, conventional immunoassays such as ELISA, Western blots, sandwich assays and the like are typically used to assay a single pre-determined analyte, e.g., a single protein of interest. While it is possible to multiplex these assays, multiplexing is severely limited by the lack of suitable distinguishable labels. As such, conventional immunoassays if they are multiplexed, are only suitable for assaying for a very small number, e.g., two or three, analytes of interest.

Further, although immunoassays could, in theory, be performed in parallel to simultaneously analyze several analytes in a sample, parallel analysis would be impractical because the assays would require a significant amount of time, cost and effort. Performing several immunoassays in parallel also requires dividing a sample between all of the individual assays, an option that is not always available. As such, immunoassays are not practical for simultaneous analysis of several analytes in a sample.

Another current methodology that, so far, has been unsuitable for the simultaneous analysis of several analytes in a complex sample is mass spectrometry. Many biological samples are complex in that they contain tens of thousands, if not millions, of analytes. Typical mass spectrometers are unable to resolve all of the analytes of such samples because a signal from an analyte of interest may be masked by a signal from another analyte, making it impossible to assess the presence of the analyte of interest with any accuracy. Mass spectrometers, alone, are inherently unsuitable for the analysis of complex samples since they cannot adequately distinguish between the analytes of the complex samples. As a result of this, mass spectrometers are usually used in conjunction with other analyte separation devices such as gas chromatography or HPLC devices in order to separate analytes prior to their analysis in the mass spectrometer. Combining other analyte separation methods with mass spectrometry solves many of the inherent problems of mass spectrometry, but, because analyte separation equipment cannot systematically separate analytes of interest (which may be analytes having diverse biochemical or physical properties such as known components of any biochemical or signal transduction pathway) away from those that are not of interest, the mass spectrometer, so far, has found little use in simultaneous analysis of several analytes in a sample.

Accordingly, while there is a great need for methods for simultaneously analyzing several constituents of a complex sample, conventional methodologies fail to meet this need. The present invention combines novel affinity-based analyte separation methods with mass spectrometry and meets this need, and others.

Relevant Literature

References of interest include: published U.S. patent applications 20010019829, 20010014461, 20020137106, 20020142343, 20020150927, 20020155509, 20020177242, 20020182649, 20020195555, 20030077616, 20030096224, 20030219731 and 20030027216; U.S. Pat. Nos. 6,630,358, 6,365,418 6,569,383 and 6,197,599; and Neubert et al, Anal. Chem. (2002) 74:3677-3683.

### SUMMARY OF THE INVENTION

The invention provides methods for preparing a MALDI sample plate. In general, the methods involve contacting a sample with an array of features containing capture agents that specifically bind to analytes in the sample, processing any analytes bound to the capture agents for MALDI analysis, and transferring the processed analytes to a MALDI sample plate. Also provided is a system for preparing a MALDI sample plate, containing an automatic fluid delivery device that is fluidically connected to a sample, and also to MALDI processing reagents. In certain embodiments, the analytes present on the prepared subject MALDI sample plate may be evaluated by mass spectrometry. Kits and other compositions are provided for performing the subject methods. The subject invention finds use in methods of simultaneously assessing the presence of several analytes in a single sample, and, as such, the invention finds use in a variety of different medical, research and proteomics applications.

### BRIEF DESCRIPTION OF THE FIGURES

FIG. 1 is a schematic representation of an embodiment of the subject invention.

FIG. 2 is a schematic representation of another embodiment of the subject invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “sample” as used herein relates to a material or mixture of materials, typically, although not necessarily, in fluid form, e.g., aqueous, containing one or more components of interest. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

Components in a sample are termed “analytes” herein. In many embodiments, the sample is a complex sample containing at least about 10², 5×10², 10³, 5×10³, 10⁴, 5×10⁴, 10⁵, 5×10⁵, 10⁶, 5×10⁶, 10⁷, 5×10⁷, 10⁸, 10⁹, 10¹⁰, 10¹¹, 10¹² or more species of analyte.

The term “analyte” is used herein to refer to a known or unknown component of a sample, which will specifically bind to a capture agent on a substrate surface if the analyte and the capture agent are members of a specific binding pair. In general, analytes are biopolymers, i.e., an oligomer or polymer such as an oligonucleotide, a peptide, a polypeptide, an antibody, or the like. In this case, an “analyte” is referenced as a moiety in a mobile phase (typically fluid), to be detected by a “capture agent” which, in some embodiments, is bound to a substrate, or in other embodiments, is in solution. However, either of the “analyte” or “capture agent” may be the one which is to be evaluated by the other (thus, either one could be an unknown mixture of analytes, e.g., polypeptides, to be evaluated by binding with the other).

A “biopolymer” is a polymer of one or more types of repeating units, regardless of the source. Biopolymers may be found in biological systems and particularly include polypeptides and polynucleotides, as well as such compounds containing amino acids, nucleotides, or analogs thereof. The term “polynucleotide” refers to a polymer of nucleotides, or analogs thereof, of any length, including oligonucleotides that range from 10-100 nucleotides in length and polynucleotides of greater than 100 nucleotides in length. The term “polypeptide” refers to a polymer of amino acids of any length, including peptides that range from 6-50 amino acids in length and polypeptides that are greater than about 50 amino acids in length.

In most embodiments, the terms “polypeptide” and “protein” are used interchangeably. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components, that while typically not attached in their native state, typically are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like.

In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 50 amino acids. In some embodiments, peptides are between 5 and 30 amino acids in length.

The term “capture agent” refers to an agent that binds an analyte through an interaction that is sufficient to permit the agent to bind and concentrate the analyte from a homogeneous mixture of different analytes. The binding interaction is typically mediated by an affinity region of the capture agent. Typical capture agents include any polypeptides, however antibodies are usually employed. Capture agents usually “specifically bind” one or more analytes.

Accordingly, the term “capture agent” refers to a molecule or a multi-molecular complex which can specifically bind an analyte, e.g., specifically bind an analyte for the capture agent, with a dissociation constant (K_{D}) of less than about 10⁻⁶ M without binding to other targets.

The term “specific binding” refers to the ability of a capture agent to preferentially bind to a particular analyte that is present in a homogeneous mixture of different analytes. Typically, a specific binding interaction will discriminate between desirable and undesirable analytes in a sample, typically more than about 10 to 100-fold or more (e.g., more than about 1000- or 10,000-fold). Typically, the affinity between a capture agent and analyte when they are specifically bound in a capture agent/analyte complex is characterized by a K_{D} (dissociation constant) of at least 10⁻⁶ M, at least 10⁻⁷ M, at least 10⁻⁸ M, at least 10⁻⁹ M, usually up to about 10⁻¹⁰ M.

The term “capture agent/analyte complex” is a complex that results from the specific binding of a capture agent with an analyte, i.e., a “binding partner pair”. A capture agent and an analyte for the capture agent will typically specifically bind to each other under “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens, are well known in the art (see, e.g., Harlow and Lane (Antibodies: A Laboratory Manual Cold Spring Harbor Laboratory, Cold Spring Harbor, N.Y. (1989)). Conditions suitable for specific binding typically permit capture agents and target pairs that have a dissociation constant (K_{D}) of less than about 10⁻⁶ M to bind to each other, but not with other capture agents or targets.

As used herein, “binding partners” and equivalents refer to pairs of molecules that can be found in a capture agent/analyte complex, i.e., exhibit specific binding with each other.

The phrase “surface-bound capture agent” refers to a capture agent that is immobilized on a surface of a solid substrate, where the substrate can have a variety of configurations, e.g., a sheet, bead, or other structure, such as a plate with wells. In certain embodiments, the collections of capture agents employed herein are present on a surface of the same support, e.g., in the form of an array.

The term “pre-determined” refers to an element whose identity is known prior to its use. For example, a “pre-determined analyte” is an analyte whose identity is known prior to any binding to a capture agent. An element may be known by name, sequence, molecular weight, its function, or any other attribute or identifier. In some embodiments, the term “analyte of interest”, i.e., an known analyte that is of interest, is used synonymously with the term “pre-determined analyte”.

The terms “antibody” and “immunoglobulin” are used interchangeably herein to refer to a capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)). Monoclonal antibodies and “phage display” antibodies are well known in the art and encompassed by the term “antibodies”.

The term “mixture”, as used herein, refers to a combination of elements, e.g., capture agents or analytes, that are interspersed and not in any particular order. A mixture is homogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of capture agents, as is commonly known in the art and described below, is not a mixture of capture agents because the species of capture agents are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

By “remote location,” it is meant a location other than the location at which the mass spectrometer is present and binding occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

The term “array” encompasses the term “microarray” and refers to an ordered array of capture agents for binding to aqueous analytes and the like.

An “array,” includes any two-dimensional or substantially two-dimensional (as well as a three-dimensional) arrangement of spatially addressable regions (i.e., “features”) containing capture agents, particularly antibodies, and the like. Where the arrays are arrays of proteinaceous capture agents, the capture agents may be adsorbed, physisorbed, chemisorbed, or covalently attached to the arrays at any point or points along the amino acid chain. In some embodiments, the capture agents are not bound to the array, but are present in a solution that is deposited into or on features of the array.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm2, e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations. The term “array” encompasses the term “microarray” and refers to any one-dimensional, two-dimensional or substantially two-dimensional (as well as a three-dimensional) arrangement of spatially addressable regions, usually bearing biopolymeric capture agents, e.g., polypeptides, nucleic acids, and the like.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained capture agent.

An array is “addressable” when it has multiple regions of different moieties (e.g., different capture agent) such that a region (i.e., a “feature” or “spot” of the array) at a particular predetermined location (i.e., an “address”) on the array will detect a particular sequence. Array features are typically, but need not be, separated by intervening spaces.

The subject array may be an array of features, each feature corresponding to a “fluid-retaining structure”, e.g., a well, wall, hydrophobic barrier, or the like. Such arrays are well known in the art, and include 24-well, 48-well, 96-well, 192-well, 384-well and 1536-well microtiter plates, or multiple thereof. In certain embodiments, the features are delineated by a hydrophobic chemical boundary, and, accordingly, the array substrate may be planar and contain features containing a hydrophobic boundary. Features may be delineated by drawing lines between them with a hydrophobic pen (e.g., a PAP PEN from Newcomer Supply, Middleton, Wis.), for example. Other fluid retaining structures are well known in the art and include physical and chemical barriers. On one embodiment, the fluid retaining structure is formed by a bead of hydrophobic material, e.g., a bead of a viscose silicone material, around a fluid-retaining area. Capture agents may be present in the fluid retaining structure, but not necessarily bound to the surface of the array substrate.

An “array layout” refers to one or more characteristics of the features, such as feature positioning on the substrate, one or more feature dimensions, and an indication of a moiety at a given location.

The term “MALDI mass spectrometer” refers to a mass spectrometer which uses a laser as a means to desorb, volatize, and ionize an analyte.

A “MALDI sample plate” is a device that, when positionally engaged in an interrogatable relationship to a laser desorption ionization source of a MALDI mass spectrometer, can be used to deliver ions derived from an analyte on the plate to the mass spectrometer. In other words, the term “MALDI sample plate” refers to a device that is removably insertable into a MALDI mass spectrometer and contains a substrate having a surface for presenting analytes for detection by the mass spectrometer. As will be described in greater below, a MALDI sample plate may contain a plurality of features, i.e., discrete, addressable regions, each containing a different analyte for ionization by the laser of the MALDI mass spectrometer. Other references may refer to a MALDI sample plate, as used herein, as a “target” or a “probe”.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

### DETAILED DESCRIPTION OF THE INVENTION

The invention provides methods for preparing a MALDI sample plate. In general, the methods involve contacting a sample with an array of features containing capture agents that specifically bind to analytes in the sample, processing any analytes bound to the capture agents for MALDI analysis, and transferring the processed analytes to a MALDI sample plate. Also provided is a system for preparing a MALDI sample plate, containing an automatic fluid delivery device that is fluidically connected to a sample, and also to MALDI processing reagents. In certain embodiments, the analytes present on the prepared subject MALDI sample plate may be evaluated by mass spectrometry. Kits and other compositions are provided for performing the subject methods. The subject invention finds use in methods of simultaneously assessing the presence of several analytes in a single sample, and, as such, the invention finds use in a variety of different medical, research and proteomics applications.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In further describing the subject invention, the subject methods and systems for preparing a MALDI sample plate are described first, followed by a description of methods for analyzing a sample in which the sample plates find use. Finally, kits and programming, for use in practicing the subject methods are described.

Methods of Making a MALDI Sample Plate

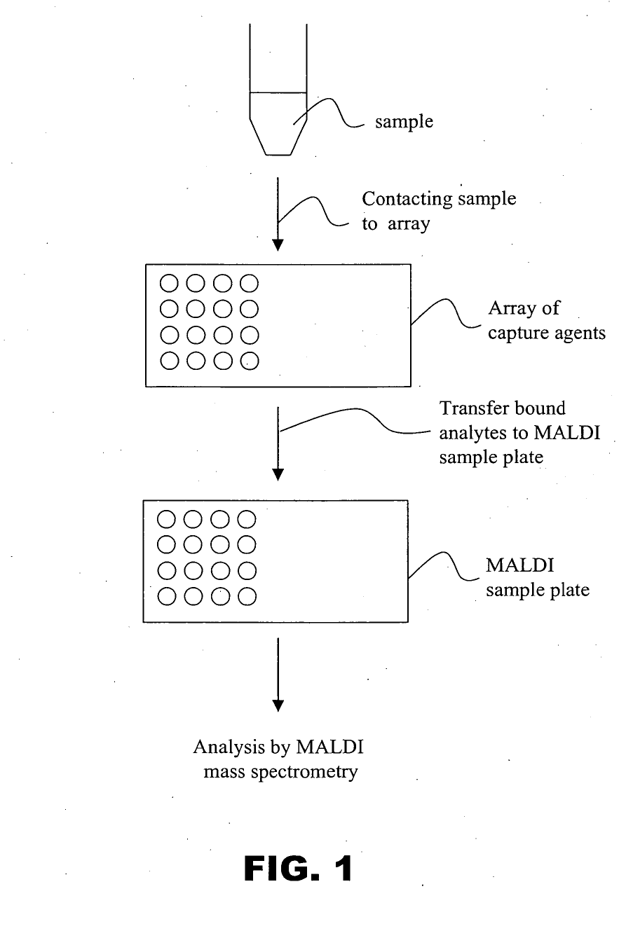

The invention provides a method for preparing a MALDI sample plate. In general, and with reference to FIG. 1, the method involves contacting a sample with an array of features, each feature containing a different surface-bound capture agent, and transferring any analytes bound to the features from the array to features of a MALDI sample plate. The array and the MALDI sample plate are different entities. In describing these methods, the arrays of capture agents and MALDI sample plates will be described first, followed by a review of the methods of making a MALDI sample plate.

Arrays of Capture Agents

The subject invention involves an array of capture agents. As described above, such an array generally comprises a plurality of spatially addressable features (e.g., more than about 10, more than about 100, more than about 500, more than 1000, features, usually up to about 10,000 or more features), and these features contain capture agents. In many embodiments, a single species of capture agent is present in each of the features, however, in other embodiments, a feature may contain a mixture of different capture agents.

In certain embodiments of the invention, the capture agents are proteinaceous capture agents, methods for the making of which are generally well known in the art. For example, polypeptides may be produced in bacterial, insect or mammalian cells (see, e.g. Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.) using recombinant means, isolated, and deposited onto a suitable substrate.

Capture agents may be selected based on their binding to predetermined analytes in a sample. Accordingly, in the subject methods, the pre-determined analytes and the capture agents that bind those analytes are selected prior to starting the subject methods. In other embodiments, the capture agents are not pre-determined and their binding specificity may be unknown.

Capture agents may be chosen using any means possible. For example, sets of capture agents present on an array may bind to proteins of a particular signal transduction, developmental or biochemical pathway, proteins having similar biological functions, proteins of similar size or structure, or they may bind proteins that are known markers for a biological condition or disease. Capture agents may also be chosen at random, or on the availability of capture agents, e.g., if a capture agent is available for purchase, for example. In some embodiments, a capture agent may be chosen purely because it is desirable to know whether a known or unknown binding partner for that capture agent is present in a sample. The binding partner for a capture agent does not have to be known for the capture agent to be present on an array for use in the subject methods.

In many embodiments, a single capture agent will bind to a single analyte. Accordingly, a set, i.e., a plurality, of capture agents for analysis is chosen. In most embodiments, each of these capture agents binds to a single species of binding partner. In other words, since an array of capture agents usually contains more than about 4, more than about 8, more than about 12, more than about 24, more than about 48, more than about 96, more than about 192, or more than about 384 or more features containing different capture agents, a corresponding number of different analytes may be present or may be suspected of being present in the sample to be assessed. In many embodiments, there are about 50-500 different capture agents on a subject array.

Further, since the capture agents are chosen using any means possible, there is no requirement that any or all of the analytes for those capture agents are present in a sample to be analyzed. In fact, since the subject methods may be used to determine the presence or absence of an analyte in a sample, as well as the level of an analyte in a sample, only a fraction or none of the analytes may be present in a sample to be analyzed.

In general, an array for use in certain embodiments of the subject methods will include at least two different features containing the same capture agent. As will be discussed in greater detail below, in these embodiments, sample is usually contacted to a fraction, but not all (e.g., one) of those features, and the uncontacted features become a “control” for the contacted features.

In particular embodiments, capture agents are monoclonal antibodies, although any molecule that can specifically bind other moieties, e.g., other types of proteins, such as members of known binding partner pairs, antibodies such as phage display antibodies and the like, may be used. Monoclonal antibodies that specifically bind to analytes are well known in the art and may be made using conventional technologies (see, e.g., Harlow and Lane, Antibodies: A Laboratory Manual Cold Spring Harbor Laboratory, Cold Spring Harbor, N.Y. (1989)). Monoclonal antibodies that specifically bind to known analytes may also be purchased from a number of antibody suppliers such as Santa Cruz Biotechnology, Santa Cruz, Calif. and Epitomics, Inc., Burlingame, Calif.

Depending on the method used, at the time at which the an array of capture agents is contacted with a sample, the capture agents may be in aqueous solution or attached, directly or via a linker, covalently or non-covalently, to a solid support. Solid supports are known in the art and include, but are not limited to, beads (e.g., magnetic or paramagnetic beads, polystyrene beads, and the like); membranes; and matrices such as agarose, sepharose and the like. Well-known solid supports include glass, polystyrene, polypropylene, polyethylene, dextran, nylon, amyloses, natural and modified celluloses, polyacrylamides, agaroses, and magnetite, and the like. In particular embodiments matrices used in immunochromatography, e.g. agarose, SEPHAROSE™-brand chromatography medium, etc., may be used. See, Scopes, 1984, Protein Purification: Principles and Practice, Springer-Verlag New York, Inc., NY, Livingstone, 1974, Methods Enzymology: Immunoaffinity Chromatography of Proteins 34:723-731).

Attachment of a capture agent to a solid support may be facilitated by using a solid support that is coated with an agent that binds to the capture agent. For example, a solid support may be coated with an antibody-binding agent such as protein A or protein G, or any other agent, e.g., streptavidin, avidin, glutathione, maltose, etc., that can bind a suitable capture agent, e.g., a biotinylated capture agent or a capture agent containing a GST, His-tag or MPB moiety. Binding capture agents to solid supports using a variety of cross-linkers is also well known in the art, and is described in great detail on pages 319-330 of Harlow and Lane (Using Antibodies: A Laboratory Manual, CSHL Press, 1999). The binding of capture agents to solid supports may provide an immunoaffinity substrate, which substrates are known in the art.

Methods for making and using arrays of polypeptides are generally well known in the art (see e.g., U.S. Pat. Nos. 6,372,483, 6,352,842, 6,346,416 and 6,242,266 MacBeath and Schreiber, Science (2000) 289:1760-3) and do not need to be described here in any more detail.

In certain embodiments, capture agents may be present in the wells of a multi-well plate, e.g., a 96-well or 384-well microtiter plate, although any solid substrate, planar or with fluid-retaining structures, may be used.

MALDI Sample Plates

In general, the MALDI sample plates, as employed in the subject methods, contain a plurality of fluid retaining structures. The area on the surface of the MALDI sample plate defined by those structures are termed “features” herein.

Typically, the number of fluid retaining structures present on a MALDI sample plate ranges from about 1 to about 2000 or more, for example as many as about 2500, 3000, 3500, 4000, 4500, and 5000 or more fluid retaining structures may be present on a single plate. The configuration or pattern of fluid retaining structures may vary depending on the particular MALDI protocol being employed, the number of fluid retaining structures present, the size and shape of the fluid retaining structures present, in certain embodiments the size, shape and pattern of the arrays to which the fluid retaining structures are to be joined, etc. For example, the pattern of the fluid retaining structures may be in the form of a grid or other analogous geometric or linear pattern or the like, e.g., similar to a conventional microtiter plate grid pattern and in certain embodiments the fluid retaining structures are present in a non grid-like or non-geometric pattern.

In general, the plates themselves may be any shape, and the choice of shape is generally defined by the shapes acceptable to the mass spectrometer chosen to be employed in the subject methods. In particular embodiments, square, rectangular, and circular plates may be used, with features arranged in a parallel, random, spiral, grid configuration or any other configuration that can be accommodated.

In general, MALDI sample plates with a plurality of fluid retaining structures are known and described in U.S. Patent Publication serial nos. 20030057368, and 20030116707. For example, e.g., “anchor” sample plates that have hydrophobic and/or hydrophilic coatings (see, e.g., U.S. Pat. No. 6,287,872) are well known and purchasable in 96 sample and 384 sample formats from Bruker Daltonik (Germany). Other suitable MALDI sample plates are purchasable from Agilent Technologies (Palo Alto, Calif.).

Methods for Making a MALDI Sample Plate

Upon selection of a set of capture agents and their placement in an array, a sample, which is usually an aqueous sample, is contacted with the capture agents under conditions suitable for specific binding of analytes in the sample to the capture agents. Specific binding conditions for most conceivable capture agent/analyte interactions are well known in the art and generally involve incubating the capture agent/analyte mixture in a binding buffer, e.g., phosphate buffered saline (PBS; 137 mM NaCl, 10 mM phosphate, 2.7 mM KCl, pH 7.4) or Tris buffered saline (10 mM Tris 50 mM NaCl, pH. 7.0) for a period of time, usually from 1 to 12 hours.

In general, many embodiments of the instant methods may also involve a separation step, e.g., a washing step where any analytes that are not specifically bound to capture agents are washed away and usually discarded. Washing may be done in binding buffer, as described above. If the capture agents are in solution, the capture agents are usually bound to a solid support prior to any washing to prevent them from being washed away. In the cases where the solid support forms a slurry or the like, the solid support may be pelleted by magnetism or centrifugation to prevent it from being washed away. Alternatively, a filter may be used.

Upon contacting a sample with a mixture of capture agents under conditions suitable for specific binding of the analytes in the sample to the capture agents, capture agent/analyte complexes are formed if analytes corresponding to the capture agents are present in the sample. As discussed above, it is not required that any complexes form since the analytes may not be in the sample tested.

In certain embodiments, after capture agent/analyte complexes are formed, bound analytes are not eluted from the capture agent and the entire capture agent/analyte complex is prepared for MALDI analysis and then transferred to a MALDI sample plate.

In other embodiments, after capture agent/analyte complexes are formed, the analytes bound in capture agent/analyte complexes are separated, e.g., eluted, from the capture agents to become free in solution. This is usually done by incubating the capture agent/analyte complexes under conditions suitable for separation of capture agent and analyte of a capture agent/analyte complex. Such conditions vary depending on the type of capture agent used and how it may be bound to a solid support, and generally involve incubating the complexes in an elution buffer that has high pH (e.g., pH 11-13), low pH (e.g., pH 1-4), high salt (e.g., 5 M LiCl or 3.5 M MgCl₂), ionic detergents (e.g., 1% SDS), dissociating agents (e.g., urea or guanidine HCl), chaotropic agents (e.g., thiocyanate), organic solvents (e.g., dioxane) or water, for a period of time. Elution methods for immunoaffinity protocols are very well known in the art and generally described on pages 335-339 of Harlow and Lane, supra.

After elution, the eluted analytes may be transferred directly to a MALDI sample plate, or, in other embodiments, the eluted analytes may be transferred to another substrate and prepared for MALDI analysis and then transferred to a MALDI sample plate. In these embodiments, the array may be re-used for the assessment of another sample.

In any of these embodiments, the subject analytes are usually processed for MALDI, i.e., “prepared for MALDI analysis” prior to transfer to the sample plate, using “MALDI processing reagents”. MALDI processing reagents include cleavage reagents, derivatization reagents, and matrix.

In many embodiments, in preparation for analysis by MALDI mass spectrometry, the subject analytes are cleaved, i.e., fragmented using a cleavage reagent, e.g., a chemical reagent, enzyme, or energy input, to result in at least one analyte fragment. A fragment can result from a sequence-specific or sequence independent cleavage event. Examples of reagents commonly used for cleaving polypeptides include enzymes, for example, proteases, such as thrombin, trypsin, chymotrypsin and the like, and chemicals, such as cyanogen bromide, acid, base, and o-iodobenzoic acid. A fragment can also be generated by collision induced dissociation (CID). Furthermore, a fragment can also result from multiple cleavage events such that a truncated polypeptide resulting from one cleavage event can be further truncated by additional cleavage events. In other words, an analyte may be cleaved using a combination of cleavage reagents and conditions.

Analytes may also be covalently modified (e.g., oxidized, de-phosphorylated, de-sulphonated, de-carboxylated, alkylated, reduced or have any covalently bound moieties, such as carbohydrate and sulfhydryl groups removed) using derivitization reagents. As is known in the art, a wide variety of derivatization reagents are commonly used to prepare analytes for mass spectrometry with 2-sulfobenzoic acid cyclic anhydride, chlorosulfonylacetyl chloride, formic acid, p-chloromercurybenzoate, iodoacetic acid, N-ethylmaleimide, sulfonic acid and 5,5-dimethyl-1,3-cyclohexanedione being examples.

Prior to their analysis, analytes are typically mixed with an energy absorbing molecule, i.e., a matrix, as is known in the art. The matrix is typically a small organic, volatile compound with certain properties that facilitate the performance of MALDI. Accordingly, a matrix is selected based on a variety of factors such as the analyte of interest (such as type, size, and the like), etc. Examples of matrices include, but are not limited to, sinapinic acid (SA) and derivatives thereof; cinnamic acid and derivatives thereof such as alpha-cyano-4-hydroxycinnamic acid (HCCA); 2,5-dihydroxybenzoic acid (DHB); 3-hydroxypicolinic acid (HPA); 2′,4′,6′ -trihydroxyacetophenone; and dithranol. The matrix is typically dissolved in a suitable solvent that is selected at least in part so that it is miscible with the analyte solution. For example, in the analysis of peptides/proteins HCCA and SA work best with ACN/0.1% TFA as solvent and in the analysis of oligonucleotides HPA and ACN/H₂O may be employed.

After the matrix and analyte (which may be derivatized and/or fragmented) are mixed, the analyte/matrix mixture is transferred, i.e., spotted to a feature of a MALDI sample plate, and dried to form crystals.

In certain embodiments, a MALDI-processed analyte of a single feature of the array is transferred to a single feature of the sample plate. Accordingly, there is usually no mixing of analytes from different array features in the subject methods. In certain embodiments, the configuration of the array of features (e.g., the spatial relationship between the features of the array) is reproduced in the sample plate, i.e., the bound analytes have the same spatial pattern in the array and the sample plate. Accordingly, if the capture agent array is a 96-feature array, then the MALDI sample plate may be a 96-feature MALDI sample plate, and the bound analytes are transferred from the a feature in the array to a spatially equivalent feature in the sample plate. However, in many embodiments, there is no spatial relationship between the features of the array and the features of the sample plate, and the analytes may be deposited in any order, in any feature.

As mentioned above, in certain embodiments, the same capture agent may be present in two different features of the array. In these embodiments, one of those features will typically be contacted with sample, whereas the other will not be contacted with sample. If a MALDI processing step is performed on these features, then the processed products (e.g., tryptic peptides from the enzymatic digestion) from those features are usually both transferred to a MALDI sample plate. Accordingly, a sample plate may contain two features of processed products, one containing a particular antibody that is enzymatically digested, and the other containing a mixture of antibody and a bound analyte, both enzymatically digested.

Accordingly, the method includes the following steps: a) contacting a sample with an array of features containing capture agents; b) processing any analytes bound to the capture agents for MALDI analysis; and c) transferring any products from the previous step from the array to features of a MALDI sample plate. In certain embodiments, the first two steps may employ a single fluid delivery device, which, as will be explained in greater detail below, may be a pulse-jet fluid delivery device or a contact fluid delivery device. This device, in certain embodiments, may also be employed to fabricate the array of capture agents used in the subject methods, and may also be employed to perform step c) of the above method.

Accordingly, a MALDI sample plate may be produced using the subject methods.

In one particular embodiment, each of the features on the array contains different capture agents, and a single sample is contacted with each of those features. In this case, the resulting MALDI sample plate will usually contain a plurality of features containing different analytes. In other embodiments, each of the features on the array contains the same capture agent, and different samples are contacted with those features. In this case, the resulting MALDI sample plate will usually contain a plurality of features containing the same analyte, if it is present in all of the samples used.

System for Preparing a MALDI Sample Plate

The invention provides a system for preparing analytes for analysis by mass spectrometry. In general, the subject system contains an automated fluid delivery device that is fluidically connected to a) an aqueous sample containing analytes, and b) MALDI processing reagents, as discussed above. In most embodiments, the system can sequentially deposit the sample and the processing reagents onto an array of capture agents. In general, the system may be employed to: contact a sample with features of an array; and contact the same features of the array with MALDI-processing reagents to prepare any bound analytes for MALDI analysis. The processed, bound analytes may then be transferred to a MALDI sample plate using the same fluid delivery device. The analytes on the sample plate are then crystallized to form a MALDI sample plate containing analytes that is suitable for use in a MALDI mass spectrometer.

Optionally, such a system may be fluidically connected to an aqueous solution of capture agents, so that that the system, prior to depositing sample on the array, may first deposit capture agents onto features of the array.

As will be discussed below, the above system may be used to assess analytes in a sample, and, accordingly, the system may further include a MALDI mass spectrometer.

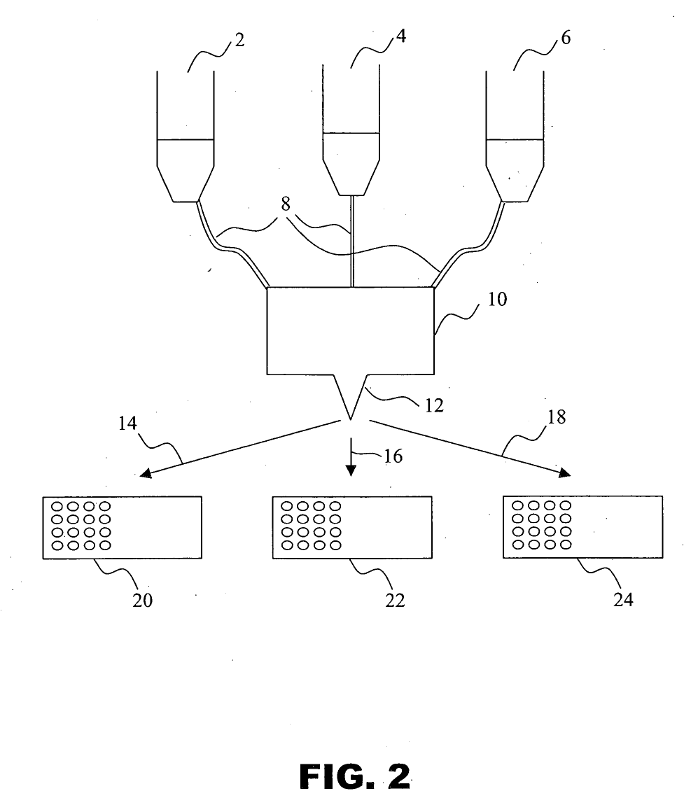

The methods may be performed by hand. However, in certain embodiments, the subject methods may be performed using an automated system, i.e., an automated fluid delivery device. An exemplary system for preparing analytes by mass spectrometry is shown in FIG. 2. The system generally contains a fluid delivery device 10 with at least one fluid delivery head 12 (e.g., a nozzle, spray, tip, pipette, or the like), fluidically linked (by means of elements 8, e.g., a capillary or the like) to one or more vessels containing sample 4, one or more vessels containing MALDI processing reagent 6, and optionally, one or more vessels containing capture agents 2.

It is recognized that the device may contain multiple fluid delivery heads or a single fluid head. In certain embodiments, therefore, the elements indicated as 8 may each fluidically connect to a different fluid delivery head, and, in other embodiments, the elements indicated by 8 may each fluidically connect to the same fluid delivery head.

In use, the device delivers reagents to features of an array in sequential order. Starting with the optional capture agent delivery step, the device will first optionally deliver capture agents to features of an array 14 to make an array of capture agent features 20. The device then delivers sample to an array of capture agents 16 to produce an array of targets containing capture agent and sample 22. Finally, the device delivers MALDI processing reagents to the array of targets containing capture agent and sample 18 in order to produce an array of “MALDI-processed analytes”, i.e., analytes that have been processed for analysis by MALDI mass spectrometry 24.

Optional washing steps may be performed by the subject device, or a different device, and the subject reagents are usually delivered in a buffer suitable for their use. Suitable incubation steps may intervene the automated steps, as described above, and, if the capture agents are not already immobilized to the surface of the array substrate at the beginning of the method, they can be immobilized prior to any washing steps. The subject system may therefore be combined with suitable means, e.g., an electromagnet whenever the capture agents are immobilized onto paramagnetic particles.

Suitable fluid delivery devices include pulse-jet printing devices, and contact printing devices such as pipetting robots and the like. Suitable pipetting robots usually perform all of the steps described above (including providing an array of capture agents) and include the following systems: GENESIS™ or FREEDOM™ of Tecan (Switzerland), MICROLAB 4000™ of Hamilton (Reno, Nev.), QIAGEN 8000™ of Qiagen (Valencia, Calif.), the BIOMEK 2000™ of Beckman Coulter (Fullerton, Calif.) and the HYDRA™ of Robbins Scientific (Hudson, N.H.). In particular embodiments, pulse-jet printing devices such as piezoelectric devices may be used (see e.g., Li et al., J. Proteome Res. (2002) 1:537-547; Sloan et al., Molecular and Cellular Proteomics (2002) 490-499).

Since fluid delivery devices can have multiple fluid delivery heads, and each head can deliver the same of different fluids, the above methods may be performed by simultaneously delivering liquids. For example, capture agent, sample, MALDI-processing agents, buffers, etc., may be simultaneously deposited (i.e., at the same time, in parallel) onto a plurality (e.g., 2, 4, 8, 12, 24, 48, 96, or a multiple thereof) of the features of an array, or a plurality of MALDI-processed samples may be simultaneously transferred from the array to a MALDI sample plate.

During transfer, a MALDI-processed sample may be concentrated by employing a suitable sample-concentration system, e.g., a molecular weight cut-off filter, or an appropriate chromatography resin. In certain embodiments, a transfer vessel (e.g., a pipette tip) containing a resin may be employed. As is known in the art, after digestion, a digested sample may be drawn into such a vessel, the analytes bound by the resin, and analytes eluted onto the MALDI sample plate. In certain embodiments, the analytes may be eluted in MALDI matrix.

In most embodiments, the subject robotic device may be programmed to perform the subject methods.

Methods of Assessing a Sample

A subject MALDI sample plate, methods for the making of which are described above, may be inserted into the MALDI source of a mass spectrometer and used to assess the analytes that are present in the features of the plate. Accordingly, the invention provides a method for assessing a sample. In general the methods involve contacting a sample with an array of features containing capture agents for analytes of interest, transferring any analytes bound to the capture agents of the features to a MALDI sample plate, and evaluating the transferred analytes using MALDI mass spectrometry.

Accordingly, the analytes isolated using the array of capture agents are evaluated using mass spectrometry. As discussed above, the analytes may be directly analyzed, or, in other embodiments, the isolated analytes may be digested into fragments prior to analysis. Accordingly, the subject isolated analytes may be intact or fragmented (i.e., digested with an enzyme) prior to their analysis in a mass spectrometer.

The isolated analytes are analyzed using any mass spectrometer that has the capability of measuring analyte, e.g., polypeptide, masses with high mass accuracy, precision, and resolution. Accordingly, the isolated analytes may be analyzed by any one of a number of mass spectrometry methods, including, but not limited to, matrix-assisted laser desorption ionization time-of-flight mass spectrometry (MALDI-TOF) and any tandem MS such as QTOF, TOF-TOF, etc.). In many embodiments, the isolated analytes are usually concentrated on the MALDI sample plate using standard technology, e.g., repeated sample spotting followed by evaporation, to a suitable concentration, e.g., 1-10 pMol/μl.

Mass spectrometry methods are generally well known in the art (see Burlingame et al. Anal. Chem. 70:647R-716R (1998); Kinter and Sherman, Protein Sequencing and Identification Using Tandem Mass Spectrometry Wiley-Interscience, New York (2000)). The basic processes associated with a mass spectrometry method are the generation of gas-phase ions derived from the sample, and the measurement of their mass.

In typical analytes that bind to the capture agents are usually the only analytes of interest in analytes present on a MALDI plate, and the masses of ions produced by those analytes may be calculated by methods known in the art, further techniques such as selective ion monitoring (SIM) may be employed to monitor only those ions that correspond to the analytes of interest.

The output from the above analysis contains the masses, i.e., the molecular weights, of the isolated analytes or fragments thereof, and their relative or absolute abundances in the sample.

The analyte masses obtained from mass spectrometry analysis may be compared to those expected for the analytes. By performing this comparison, any signals obtained that are not derived from the analytes of interest may be discarded, and only those signals corresponding to the pre-determined analytes may be retained. In many embodiments, the masses of the analytes or fragments thereof are stored in a table of a database and the table usually contains at least two fields, one field containing molecular mass information, and the other field containing analyte identifiers, such as names or codes. As such, the subject methods may involve comparing data obtained from mass spectrometry to a database to identify data for an analyte of interest.

In general, methods of comparing data produced by mass spectrometry to databases of molecular mass information to facilitate data analysis is very well known in the art (see, e.g., Yates et al, Anal Biochem. 1993 214:397-408; Mann et al, Biol Mass Spectrom. 1993 22:338-45; Jensen et al, Anal Chem. 1997 D69:4741-50; and Cottrell et al., Pept Res. 1994 7:115-24) and, as such, need not be described here in any further detail.

Accordingly, information, e.g., data, regarding the amount of analytes in a sample of interest (including information on their presence or absence) may be obtained using mass spectrometry.

In certain embodiments, data may be obtained from processed materials from two features of capture agents, one that has been contacted to sample and the other that has not been subjected to sample. In these embodiments, one set of data will correspond to a MALDI-processed (e.g., digested) capture agent, i.e., a capture agent that has been processed for analysis for analysis by MALDI-MS, and the other will correspond to a MALDI-processed capture agent/analyte complex, i.e., a capture agent/analyte complex that has been processed for analysis for analysis by MALDI-MS. In these embodiments, the molecular mass information corresponding to the bound analyte may be determined by removal of the molecular mass information for the capture agent from the molecular mass information for the capture agent/analyte complex. Accordingly, even if molecular mass information is obtained for both an antibody and its capture agent, the presence of the capture agent can be assessed.

As is well known in the art, for each analyte, information obtained using mass spectrometry may be qualitative (e.g., showing the presence or absence of an analyte, or whether the analyte is present at a greater or lower amount than a control analyte or other standard) or quantitative (e.g., providing a numeral or fraction that may be absolute or relative to a control analyte or other standard). Also as is known, standards for assessing mass spectrometry data may be obtained from a control analyte that is present in the isolated analytes, such as an analyte of known concentration, or an analyte that has been added at a known amount to the isolated analytes, e.g., a spiked analyte.

Accordingly, the data produced by the subject methods may be “normalized” to an internal control, e.g. an analyte of known concentration or the like.

By comparing the results from assessing the presence of an analyte in two or more different samples using the methods set forth above, the relative levels of an analyte in two or more different samples may be obtained. In other embodiments, by assessing the presence of at least two different analytes in a single sample, the relative levels of the analytes in the sample may be obtained.

Utility

The subject methods may be employed in a variety of diagnostic, drug discovery, and research applications that include, but are not limited to, diagnosis or monitoring of a disease or condition (where analytes that are markers for the disease or condition are assessed), discovery of drug targets (an analyte whose level is modulated in a disease or condition is a drug target), drug screening (where the effects of a drug are monitored by assessing the levels of analytes), protein fingerprinting (where the profile, i.e., the expression levels of analytes are assessed in a variety of diseases or artificial conditions and the profile provides a fingerprint for that disease or condition), determining drug susceptibility (where drug susceptibility is associated with a particular profile of analytes), discovery of new binding partners (where an analyte that binds to a capture agent has not been previously identified) and research (where is it desirable to know the relative concentrations of a number of analytes in a sample, or, conversely, the relative levels of an analyte in two or more samples).

In most embodiments, a sample is contacted with an array of capture agents that specifically bind to a set of pre-determined analytes in the sample under conditions suitable to produce capture agent/analyte complexes. The analytes bound in the capture agent/analyte complexes are analyzed by mass spectrometry, and, by integrating the signals produced by the ions of the analytes, measurements corresponding to the abundance of particular ions are provided. Using software that is already available and commonly used to identify ion masses, the data is usually compared to a database of ion masses expected for the analytes. By doing this comparison, the identity and abundance of a bound analyte corresponding to a particular ion becomes known. Depending on the exact method used, a table containing data on the abundance of analytes may be exported to a separate database, and saved.

Once produced, any data may be transmitted to a remote location for further evaluation and/or use. For example, the data may be transmitted and stored in a database for future use. Any convenient telecommunications means may be employed for transmitting the data, e.g., facsimile, modem, internet, etc.

In an embodiment of particular interest, a subject system is used to deliver immobilizable capture agents (e.g., capture agents linked to paramagnetic beads, or the like) to fluid-retaining structures of an array substrate. The subject system is then used to deliver sample to the fluid-retaining structures of the array substrate, and the array substrate is incubated for a period of time, usually between 1 and 12 hours to allow binding of analytes in the sample to the capture agents. Then, in certain embodiments, the capture agents are immobilized (e.g., by paramagnetism) and washed using the system. The subject system is then used to deliver MALDI processing agents (which may be done sequentially and with intermediate washing steps if more than one type of agent is to be added), to process the bound analytes for MALDI. As a final step, the system usually delivers matrix to the fluid-retaining structures, and, usually using a different device, the processed analytes are transferred to a MALDI plate and dried.

Computer-Related Embodiments

The invention also provides a variety of computer-related embodiments. Specifically, the automated means for performing the methods described above may be controlled using computer-readable instructions, i.e., programming. Accordingly, the invention provides computer programming for directing a means, e.g., a liquid handling workstation, to make a MALDI sample plate using capture agents, or an array thereof, a sample, and a MALDI sample plate that contains no analytes.

In most embodiments, the methods are coded onto a computer-readable medium in the form of “programming”, where the term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A “processor” references any hardware and/or software combination which will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

In most embodiments, the processor will be operable linkage, i.e., part of-or networked to, the aforementioned workstation, and capable of directing its activities.

Kits

Also provided by the subject invention are kits for practicing the subject methods, as described above. The subject kits at least include a set of capture agents that specifically bind to a corresponding set of analytes. As discussed above, the capture agents may be provided as an array of capture agents. Other optional components of the kit include: control analytes for spiking into a sample, buffers, including binding, washing and elution buffers, solid supports, such as beads, protein A or G or avidin coated sepharose or agarose, etc., and a MALDI sample plate. The kit may also contain a database, which may be a table, on paper or in electronic media, containing molecular mass information for the analytes to which the capture agents provided by the kit correspond. In some embodiments, the kits contain programming to allow a robotic system to perform the subject methods, e.g., programming for instructing a robotic pipettor or a contact or inkjet printer to add, mix and remove reagents, as described above. The various components of the kit may be present in separate containers or certain compatible components may be precombined into a single container, as desired.

The subject kits may also include one or more other reagents for preparing or processing an analyte sample for MALDI. The reagents may include one or more matrices, solvents, sample preparation reagents, buffers, desalting reagents, enzymatic reagents, denaturing reagents, where calibration standards such as positive and negative controls may be provided as well. As such, the kits may include one or more containers such as vials or bottles, with each container containing a separate component for carrying out a sample processing or preparing step and/or for carrying out one or more steps of a MALDI protocol.

In addition to above-mentioned components, the subject kits typically further include instructions for using the components of the kit to practice the subject methods, i.e., to prepare a MALDI sample plate and/or assess a sample. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., two or more control samples for use in testing the kit.

### EXAMPLES

The following examples are put forth so as to provide those of ordinary skill in the art with a complete disclosure and description of how to make and use the present invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Detection of Analytes Bound to Capture Agents by MALDI Mass Spectrometry

Several biotinylated peptides were used as capture agents (probes). The peptide sequences are shown in Table 1.

TABLE 1
peptides used as capture agents.

| | | Molecular |
|---|---|---|
| | | weight |
| Peptide ID | Peptide sequence | (Da) |
| SmB | Biotin-PPGMRPPPPGMRRGPPPPGMRP | 2909.6 |
| | PRP | |
| | (SEQ ID NO:1) | |
| CDC25 | Biotin-SGSGEQPLT*PVTDL | 1706.9 |
| | (SEQ ID NO:2) | |
| LD10 | Biotin-SGSGAPPTPPPLPP | 1497.7 |
| | (SEQ ID NO:3) | |
| WBP1 | Biotin-SGSGGTPPPPYTVG | 1499.6 |
| | (SEQ ID NO:4) | |
| COXG | Biotin-SGSGVLIKRRST*EL-COOH | 1808.8 |
| | (SEQ ID NO:5) | |
| Kir2.1 | Biotin-SGSGPRPLRRESEI-COOH | 1766.9 |
| | (SEQ ID NO:6) | |
| Kir2.1* | Biotin-SGSGPRPLRRES*EI-COOH | 1846.8 |
| | (SEQ ID NO:7) | |
| COXD | Biotin-SGSGVLIKRRSTEL-COOH | 1728.9 |
| | (SEQ ID NO:8) | |

In Table 1, amino acids indicated with an asterisk (*) are phosphorylated. Capture agents were immobilized to avidin beads, microtiter plates and glass slides.

The following were used as targets: GST-CAP1 (a protein having PDZ domain), GST-FBP21 (a protein having WW domain), GST-14-3-3, p60Src (a protooncogene tyrosine protein kinase known not to bind to any of the peptides listed above), and a mouse brain protein extract. Four of the 8 target peptides are proline-rich peptides, which are identified as SmB, CDC25, LD10 and WBP1, and are known to bind SH3 domains. Another set of 4 peptides (e.g., COXG, Kir2.1, Kir 2.1 phosphorylated, and COXD) are arginine rich and are known to bind PDZ domains like 14-3-3.

To make the peptide array, the wells of a multiwell (i.e., 96-well filtration plate from Millipore with a 1.2 um pore size) filtration plate were first filled with a known volume of a suspension containing Streptavidin beads (Sigma). The biotinylated peptides were added (each in a separate well) and allowed to incubate with the avidin beads for a defined time interval following which vacuum (5-6 in. Hg) was applied to remove excess liquid. The immobilized peptides were then ready to be incubated with the protein mixture (e.g., a brain extract). After incubation, the peptide-protein complexes were washed in parallel, and subsequently subjected to enzymatic digestion (following in-gel digestion protocol). After digestion directly in the membrane plate, the digests were transferred to a MALDI plate for MS analysis, after concentration using a C18 resin-containing Millipore C18 ZIPTIP™.

All experiments were conducted with Streptavidin Agarose beads (Sigma S1638) with 1-2 mg SA (streptavidin) bound/mL of resin, with a binding capacity of 125-30 ug biotin/mL resin.

Table 2 shows a matrix representing experiments carried out with the controlled proteins and give an indication of whether binding occurred or not. The headers for each of the columns of Table 2 indicate, for each experiment, the peptide used as a capture agent. These peptides correspond to the peptides set forth in Table 1. The first column of Table 2 indicates, for each experiment, the sample used. GST-CAP1, GST-FBP21, GST-14-3-3 are described above. “Brain extract” is a soluble protein extract of homogenized mouse brain, and, “QC” indicates the negative controls performed. M/z values indicate mass-to-charge ratios, and numbers in brackets indicate the size of the signal obtained, indicating the amount of the analytes present. In general, a signal size of about 1000 or more indicates significant binding.

TABLE 2

| | SmB | CDC25 | LD10 | WBP1 | COXG | Kir2.1 | Kir2.1* | COXD |
|---|---|---|---|---|---|---|---|---|
| GST- | 0 | m/z 1358.8 | m/z 1358.8 | 0 | 0 | m/z 1358.8 | m/z 1152.6 | m/z 1358.8 |
| CAP1 | | (480) | (1,600) | | | (250) | <100 | (1,100) |
| GST- | ? | m/z 1138.7 | m/z 1138.7 | m/z 1138.7 | m/z 1138.7 | m/z 1152.8 | m/z 1152.8 | m/z 1138.7 |
| FBP21 | | (500) | (15,000) | (9,000) | (30,000) | (900) | (8,000) | (200) |
| GST-14- | m/z 1245.7 | m/z 1245.7 | m/z 1245.7 | 0 | m/z 1245.7 | m/z 1245.7 | m/z 1245.7 | m/z 1245.7 |
| 3-3 | (0) | (0) | (150) | | (500) | (400) | (8,000) | (3600) |
| | m/z 1255.9 | m/z 1255.9 | | | | | | |
| | (3,000) is | (800) | | | | | | |
| | GPPPPG | | | | | | | |
| | MRPPRP | | | | | | | |
| Brain | Protein | Unidentified | Guanylate | <100 | <100 | <25 | <50 | <50 |
| extract | identified | target | kinase | | | | | |
| | as WD11 | | associated | | | | | |
| | repeat domain | | protein | | | | | |
| | protein | | (see data) | | | | | |
| QC | SmB alone digest | CDC25 on | LD10 on | WBP1 on | COXG | Kir2.1 | Kir2.1* | COXD |
| | SmB on beads (no | beads (no | beads (no | beads (no | on beads | on beads | alone digest | alone |
| | brain extract) | brain | brain | brain | (no brain | (no brain | CDC25 | digest |
| | followed by | extract) | extract) | extract) | extract) | extract) | on beads | |
| | digestion | followed | followed | followed | followed | followed | (no brain | |
| | SmB + Src | by digestion | by digestion | by digestion | by digestion | by digestion | extract) | |

As expected, none of the negative control experiments indicated in the “QC” row gave significant signals, and P60Src did not significantly bind any SmB. The SmB field of the QC row of this table indicates that three separate experiments were carried out: digestion of SmB alone, digestion of SmB captured on avidin beads (without any brain extract), and lastly digestion of SmB that was immobilized on avidin beads and then incubated with Src to demonstrate that Src does not bind to SmB. Significant binding was detected between several capture agents and several probes.

Table 3 shows results obtained using the brain extract. The spectra obtained matches almost exactly to those expected for a particular, known, WD11 repeat protein. This WD11 repeat protein binds only the SmB peptide and none of the other 7 peptides.

TABLE 3

| Experimental m/z | Experimental m/z | Theoretical |
|---|---|---|
| (Run 1) | (Run 2) | m/z |
| | 977.67232 | 977.4873 |
| | 1021.69333 | |
| 1230.70845 | | 1231.6351 |
| 1255.75744 | 1255.75078 | |
| 1287.73506 | | 1287.7340 |
| 1418.78338 | 1418.77852 | 1418.6845 |
| 1467.xxxx | 1467.82565 | |
| 1498.xxxx | 1498.82564 | |
| 1515.84330 | 1515.8206 | 1515.8206 |
| 1600.92481 | 1600.8805 | 1600.8805 |
| 1705.90494 | 1705.90494 | |
| 1709.8679 | | 1709.8679 |
| | 1727.88837 | 1727.8996 |
| 1790.97960 | 1790.9824 | |
| | 1811.97676 | 1811.8817 |

Accordingly, the methods may be used to identify new binding partners in complex protein extracts.

The above results and discussion demonstrate a new method for producing a MALDI sample plate and for analyzing components of a sample using MALDI mass spectrometry. Such methods are superior to currently used methods because they provide a way of simultaneously assessing, in parallel, several analytes in a single sample. Accordingly, as such, the subject methods represent a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for preparing a MALDI sample plate, comprising:
(a) contacting a sample with an array of features containing different capture agents;
(b) processing any analytes bound to said capture agents for MALDI analysis; and,
(c) transferring any products from step (b) to features of a MALDI sample plate, to prepare said MALDI sample plate.

2. The method of claim 1, wherein step (a) employs an automated fluid delivery device, and steps (b) and (c) employ the same automated fluid delivery device.

3. The method of claim 2, wherein said device is a pulse-jet fluid delivery device.

4. The method of claim 2, wherein said device is a contact fluid delivery device.

5. The method of claim 1, wherein said array of features is fabricated by depositing said capture agents onto a substrate using a fluid delivery device that is also employed in steps (a) and (b).

6. The method of claim 1, wherein said array comprises a substrate having fluid retaining structures.

7. The method of claim 6, wherein said substrate is a planar substrate.

8. The method of claim 1, wherein said contacting step comprises separating analytes that are bound to said capture agents from those that are not bound to said capture agents.

9. The method of claim 1, further comprising:
drying said transferred products on said MALDI sample plate.

10. The method of claim 1, wherein said capture agents are antibodies.

11. The method of claim 1, wherein said capture agents comprise an affinity label for binding to a solid support.

12. A method for assessing a sample, comprising:
performing the method of claim 1; and
(d) evaluating said transferred products using a MALDI mass spectrometer, to assess said sample.

13. The method of claim 12, wherein said evaluating is determining molecular weights of analytes bound to said capture agents.

14. The method of claim 13, further comprising comparing said obtained molecular weights to molecular weights of pre-determined analytes.

15. The method of claim 14, wherein said molecular weights for said pre-determined analytes are in a database.

16. The method of claim 12, wherein said evaluating is determining amounts of said analytes bound to said capture agents.

17. The method of claim 12, wherein said evaluating is qualitative or quantitative.

18. The method of claim 12, wherein said evaluating is assessing the formation of capture agent/analyte complexes relative to the formation of control capture agent/analyte complexes.

19. An automated system for preparing analytes for analysis by mass spectrometry, comprising:
an automated fluid delivery device that is fluidically connected to:
a sample containing said analytes; and
MALDI processing reagents;
wherein said system can sequentially deposit said sample and said agents onto the surface of an array.

20. The system of claim 19, wherein said automated fluid delivery device is further fluidically connected to:
a solution of capture agents,
wherein said system can deposit said capture agents onto the surface of an array prior to depositing said analytes and MALDI processing agents.

21. The system of claim 19, wherein said automated fluid delivery device is capable of transferring said sample and said reagents from the surface of said array to the surface of a MALDI sample plate.

22. The system of claim 20, wherein said array comprises a substrate comprising fluid retaining structures.

23. The automated system of claim 22, wherein said fluid-retaining structures comprise hydrophobic boundaries.

24. The system of claim 19, wherein said device is a robotic device.

25. The system of claim 24, wherein said device is a pulse-jet fluid delivery device.

26. The method of claim 24, wherein said device is a contact fluid delivery device.

27. The automated system of claim 19, wherein said MALDI processing reagents contain cleavage reagents or a MALDI matrix.

28. A method comprising transmitting data from a method of claim 12 from a first location to a second location.

29. The method of claim 28, wherein said second location is a remote location.

30. A method comprising receiving a transmitted result of a reading of an array obtained according to the method of claim 12.

31. A kit for analyzing a sample, comprising:
a plurality of capture agents; and
instructions for preparing a MALDI sample plate using said capture agents using the method of claim 1.

32. The kit of claim 31, wherein said captures agents are present in an array.

33. The kit of claim 31, further comprising MALDI processing reagents.

34. The kit of claim 33, wherein said MALDI processing reagents comprise at least one cleavage reagent.

35. A computer-readable medium comprising:
programming for controlling the automated system of claim 19.

36. A computer comprising the computer-readable medium of claim 34.

37. A computer implemented method for preparing a MALDI sample plate, comprising:
directing a fluid delivery device to:
(a) contact a sample with an array of features containing capture agents;
(b) process any analytes bound to said capture agents for MALDI analysis; and,
(c) transfer any products from step (b) from said array to features of a MALDI sample plate,
to prepare said MALDI sample plate.


SEQUENCE LISTING (8 sequences)

SEQ ID NO: 1 - protein, 25 aa, Artificial Sequence
  Synthetic peptide
PPGMRPPPPGMRRGPPPPGMRPPRP

SEQ ID NO: 2 - protein, 14 aa, Artificial Sequence
  Synthetic peptide
SGSGEQPLTPVTDL

SEQ ID NO: 3 - protein, 14 aa, Artificial Sequence
  Synthetic peptide
SGSGAPPTPPPLPP

SEQ ID NO: 4 - protein, 14 aa, Artificial Sequence
  Synthetic peptide
SGSGGTPPPPYTVG

SEQ ID NO: 5 - protein, 14 aa, Artificial Sequence
  Synthetic peptide
SGSGVLIKRRSTEL

SEQ ID NO: 6 - protein, 14 aa, Artificial Sequence
  Synthetic peptide
SGSGPRPLRRESEI

SEQ ID NO: 7 - protein, 14 aa, Artificial Sequence
  Synthetic peptide
SGSGPRPLRRESEI

SEQ ID NO: 8 - protein, 14 aa, Artificial Sequence
  Synthetic peptide
SGSGVLIKRRSTEL